(19) United States
(12) Patent Application Publication
(10) Pub. No.: US 2021/0041512 A1
(43) Pub. Date: Feb. 11, 2021

(54) SYSTEMS AND METHODS FOR MULTIPLEXED OR INTERLEAVED OPERATION OF MAGNETOMETERS

(71) Applicant: HI LLC, Los Angeles, CA (US)

(72) Inventors: Ethan Pratt, Santa Clara, CA (US); Jamu Alford, Simi Valley, CA (US)

(21) Appl. No.: 16/984,720

(22) Filed: Aug. 4, 2020

Related U.S. Application Data
(60) Provisional application No. 62/926,032, filed on Oct. 25, 2019.
(60) Provisional application No. 62/883,399, filed on Aug. 6, 2019.

(51) Int. Cl. G01R 33/26; A61B 5/04; A61B 5/00

(52) U.S. Cl. CPC G01R 33/26; A61B 5/04008; A61B 2562/04; A61B 2562/18; A61B 5/6803

(57) ABSTRACT

A magnetic field measurement system includes a body; sensors units that each include at least one magnetic field sensor disposed on or in the body; magnetic field generators, each of the magnetic field generators associated with a different one of the sensor units to provide active shielding when the magnetic field generator is activated; and a processor coupled to the magnetic field sensors and the magnetic field generators and configured to perform actions including: 1) selecting at least one of the sensor units, wherein, when multiple sensor units are selected, the selected sensor units are spatially separated from each other; 2) for each of the at least one selected sensor unit, activating the magnetic field generator associated with that selected sensor unit to provide active shielding; 3) receiving signals from the at least one selected sensor unit; and 4) repeating 1) through 3) at least once.

### CROSS-REFERENCE TO RELATED APPLICATIONS

This application claims the benefit of U.S. Provisional Patent Application Ser. Nos. 62/883,399, filed Aug. 6, 2019, and 62/926,032, filed Oct. 25, 2019, both of which are incorporated herein by reference in their entireties.

### FIELD

The present disclosure is directed to the area of magnetic field measurement systems including systems for magnetoencephalography (MEG). The present disclosure is also directed to methods and systems for multiplexing or interleaving operation of magnetometers in a magnetic field measurement system.

### BACKGROUND

In the nervous system, neurons propagate signals via action potentials. These are brief electric currents which flow down the length of a neuron causing chemical transmitters to be released at a synapse. The time-varying electrical current within an ensemble of neurons generates a magnetic field. Magnetoencephalography (MEG), the measurement of magnetic fields generated by the brain, is one method for observing these neural signals.

Highly sensitive magnetometers for MEG neural recording can be designed to operate in a near zero magnetic field environment. As an example, optical magnetometry is the use of optical methods to measure a magnetic field with very high accuracy—on the order of 1×10⁻¹⁵ Tesla (1 fT) and optically-pumped magnetometer (OPM) sensors are of particular interest in the measurement of biological magnetism such as magnetencephalography (MEG). One challenge with this approach is the difference in scale between the biological signals, which are on the order of 1 fT to 1 pT, and the magnetic field of the Earth, which is 20 μT to 50 μT depending on location.

In the nervous system, neurons propagate signals via action potentials. These are brief electric currents which flow down the length of a neuron causing chemical transmitters to be released at a synapse. The time-varying electrical current within the neuron generates a magnetic field, which propagates through the human body. Magnetoencephalography (MEG), the measurement of magnetic fields generated by the brain, is one method for observing these neural signals.

Existing technology for measuring MEG typically utilizes superconducting quantum interference devices (SQUIDs) or collections of discrete optically pumped magnetometers (OPMs). SQUIDs require cryogenic cooling, which is bulky, expensive, requires a lot of maintenance. These requirements preclude their application to mobile or wearable devices.

An alternative to an array of SQUIDs is an array of OPMs. For MEG and other applications, the array of OPMS may have a large number of OPM sensors that are tightly packed. Such dense arrays can produce a high-resolution spatial mapping of the magnetic field, and at a very high sensitivity level. Such OPMs sensors can be used for a wide range of applications, including sensing magnetic field generated by neural activities, similar to MEG systems.

### BRIEF SUMMARY

One embodiment is a magnetic field measurement system for measuring biosignals. The system includes a body; a plurality of sensors units, each of the sensor units including at least one magnetic field sensor disposed on or in the body; a plurality of magnetic field generators, each of the magnetic field generators associated with a different one of the sensor units, wherein each of the magnetic field generators is configured to provide active shielding to the associated sensor unit when the magnetic field generator is activated; and a processor coupled to the magnetic field sensors and the magnetic field generators, wherein the processor is configured to perform actions including: 1) selecting at least one of the sensor units, wherein, when multiple sensor units are selected, the selected sensor units are spatially separated from each other; 2) for each of the at least one selected sensor unit, activating the magnetic field generator associated with that selected sensor unit to provide active shielding; 3) receiving signals from the at least one selected sensor unit; and 4) repeating 1) through 3) at least once.

Another embodiment is a non-transitory computer-readable medium having stored thereon instructions for execution by a processor to perform actions including: 1) selecting at least one of a plurality of sensor units of a wearable device of a magnetic field measurement system, wherein, when multiple sensor units are selected, the selected sensor units are spatially separated from each other; 2) for each of the at least one selected sensor unit, activating a magnetic field generator associated with that selected sensor unit to provide active shielding; 3) receiving signals from the at least one selected sensor unit; and 4) repeating 1) through 3) at least once.

Yet another embodiment is a method for monitoring biologically generated magnetic fields, the method including: 1) selecting at least one of a plurality of sensor units of a wearable device of a magnetic field measurement system, wherein, when multiple sensor units are selected, the selected sensor units are spatially separated from each other; 2) for each of the at least one selected sensor unit, activating a magnetic field generator associated with that selected sensor unit to provide active shielding; 3) receiving signals from the at least one selected sensor unit; and 4) repeating 1) through 3) at least once.

In at least some embodiments, the actions or method further include disabling the magnetic field generator associated with the at least one selected sensor unit after receiving the signals from the at least one selected sensor unit. In at least some embodiments, repeating 1) through 3) includes 1) through 3) until a programmed termination is reached by the system. In at least some embodiments, repeating 1) through 3) includes 1) through 3) until termination by the user.

In at least some embodiments, when multiple sensor units are selected, the selected sensor units are spatially separated from each other so that active shielding at each of the selected sensor units produces a magnetic field at any of the other selected sensor units that is no greater in magnitude than an expected magnitude of the biosignal. In at least some embodiments, when multiple sensor units are selected, the selected sensor units are spatially separated from each other by at least 2 centimeters. In at least some embodiments, when multiple sensor units are selected, the selected sensor units are spatially separated from each other so that, at each of the selected sensor units, a combined magnitude of the magnetic fields of all of the active shielding arrangements of the other selected sensor units is no more than 50 nT.

In at least some embodiments, each of the sensor units consists of a single one of the magnetic field sensors. In at least some embodiments, each of the magnetic field sensors is an optically pumped magnetometer. In at least some embodiments, the magnetic field measurement system further includes passive shielding disposed in the body to provide shielding for at least one of the sensor units.

In at least some embodiments, selecting at least one of the sensor units including selecting only one of the sensor units In at least some embodiments, upon repeating 1) through 3), selecting at least one of the sensor units includes selecting at least one of the sensor units based on the received signals. In at least some embodiments, upon repeating 1) through 3), selecting at least one of the sensor units includes selecting at least one of the sensor units based on a predetermined order.

In at least some embodiments, the actions or method further include analyzing the received signals. In at least some embodiments, the body of the magnetic field measurement system is a wearable device.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic block diagram of one embodiment of a magnetic field measurement system, according to the invention;

FIG. 1B is a schematic block diagram of one embodiment of a magnetometer, according to the invention;

FIG. 2 shows a magnetic spectrum with lines indicating dynamic ranges of magnetometers operating in different modes;

FIG. 3A is a side view of one embodiment of a wearable magnetoencephalography (MEG) device with multiple magnetometers, according to the invention;

FIG. 3B is a different side view of the MEG device of FIG. 3A, according to the invention;

FIG. 4 is a flowchart of one embodiment of a method which utilizes the multiplexing or interleaving of sensor unit operation in a limited duty cycle mode, according to the invention; and

FIG. 5 is a flowchart of another embodiment of a method which utilizes the multiplexing or interleaving of sensor unit operation in a limited duty cycle mode, according to the invention.

### DETAILED DESCRIPTION

The present disclosure is directed to the area of magnetic field measurement systems including systems for magnetoencephalography (MEG). The present disclosure is also directed to methods and systems for multiplexing or interleaving operation of magnetometers in a magnetic field measurement system.

Herein the terms “ambient background magnetic field” and “background magnetic field” are interchangeable and used to identify the magnetic field or fields associated with sources other than the magnetic field measurement system and the magnetic field sources of interest, such as biological source(s) (for example, neural signals from a user's brain) or non-biological source(s) of interest. The terms can include, for example, the Earth's magnetic field, as well as magnetic fields from magnets, electromagnets, electrical devices, and other signal or field generators in the environment, except for the magnetic field generator(s) that are part of the magnetic field measurement system.

The terms “gas cell”, “vapor cell”, and “vapor gas cell” are used interchangeably herein. Below, a gas cell containing alkali metal vapor is described, but it will be recognized that other gas cells can contain different gases or vapors for operation.

The methods and systems are described herein using optically pumped magnetometers (OPMs). While there are many types of OPMs, in general magnetometers operate in two modalities: vector mode and scalar mode. In vector mode, the OPM can measure one, two, or all three vector components of the magnetic field; while in scalar mode the OPM can measure the total magnitude of the magnetic field.

Vector mode magnetometers measure a specific component of the magnetic field, such as the radial and tangential components of magnetic fields with respect the scalp of the human head. Vector mode OPMs often operate at zero-field and may utilize a spin exchange relaxation free (SERF) mode to reach femto-Tesla sensitivities. A SERF mode OPM is one example of a vector mode OPM, but other vector mode OPMs can be used at higher magnetic fields. These SERF mode magnetometers can have high sensitivity but may not function in the presence of magnetic fields higher than the linewidth of the magnetic resonance of the atoms of about 10 nT, which is much smaller than the magnetic field strength generated by the Earth.

Magnetometers operating in the scalar mode can measure the total magnitude of the magnetic field. (Magnetometers in the vector mode can also be used for magnitude measurements.) Scalar mode OPMs often have lower sensitivity than SERF mode OPMs and are capable of operating in higher magnetic field environments.

The magnetic field measurement systems, such as a biological signal detection system, described herein can be used to measure or observe electromagnetic signals generated by one or more magnetic field sources (for example, neural signals or other biological sources) of interest. The system can measure biologically generated magnetic fields and, at least in some embodiments, can measure biologically generated magnetic fields in an unshielded or partially shielded environment. Aspects of a magnetic field measurement system will be exemplified below using magnetic signals from the brain of a user; however, biological signals from other areas of the body, as well as non-biological signals, can be measured using the system. In at least some embodiments, the system can be a wearable MEG system that can be portable and used outside a magnetically shielded room.

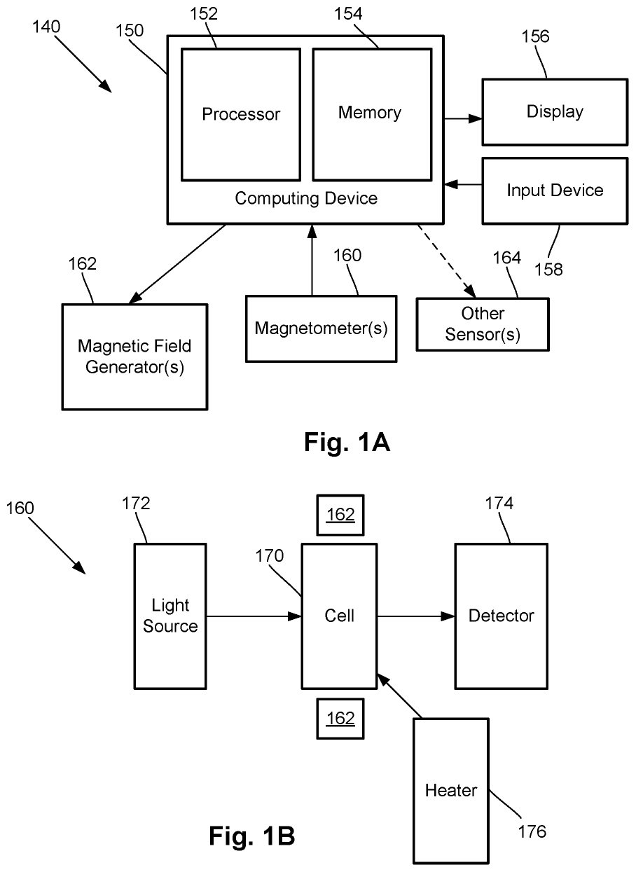

A magnetic field measurement system, such as a biological signal detection system, can utilize one or more magnetic field sensors. Magnetometers will be used herein as an example of magnetic field sensors, but other magnetic field sensors may also be used in addition to, or as an alternative to, the magnetometers. FIG. 1A is a block diagram of components of one embodiment of a magnetic field measurement system 140 (such as a biological signal detection system.) The system 140 can include a computing device 150 or any other similar device that includes a processor 152, a memory 154, a display 156, an input device 158, one or more magnetometers 160 (for example, an array of magnetometers) which can be OPMs, one or more magnetic field generators 162 (for example, shielding coil arrangements), and, optionally, one or more other sensors 164 (e.g., non-magnetic field sensors).

The systems, devices, and methods are described herein with respect to the measurement of neural signals or neural activity arising from one or more magnetic field sources of interest in the brain of a user as an example. It will be understood, however, that the system can be adapted and used to measure signals from other magnetic field sources of interest including, but not limited to, other neural signals, other biological signals (i.e., biosignals), as well as non-biological signals.

The computing device 150 can be a computer, tablet, mobile device, field programmable gate array (FPGA), microcontroller, or any other suitable device for processing information or instructions. The computing device 150 can be local to the user or can include components that are non-local to the user including one or both of the processor 152 or memory 154 (or portions thereof). For example, in at least some embodiments, the user may operate a terminal that is connected to a non-local computing device. In other embodiments, the memory 154 can be non-local to the user.

The computing device 150 can utilize any suitable processor 152 including one or more hardware processors that may be local to the user or non-local to the user or other components of the computing device. The processor 152 is configured to execute instructions stored in the memory 154.

Any suitable memory 154 can be used for the computing device 150. The memory 154 illustrates a type of computer-readable media, namely computer-readable storage media. Computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 156 can be any suitable display device, such as a monitor, screen, or the like, and can include a printer. In some embodiments, the display is optional. In some embodiments, the display 156 may be integrated into a single unit with the computing device 150, such as a tablet, smart phone, or smart watch. In at least some embodiments, the display is not local to the user. The input device 158 can be, for example, a keyboard, mouse, touch screen, track ball, joystick, voice recognition system, or any combination thereof, or the like. In at least some embodiments, the input device is not local to the user.

In at least some embodiments, the magnetic field generator(s) 162 can be used to provide active shielding. In at least some embodiments, the magnetic field generator(s) can be shielding coil arrangements with one or more coils or magnets such as, for example, Helmholtz coils, solenoid coils, planar coils, saddle coils, electromagnets, permanent magnets, or any other suitable arrangement for generating a magnetic field. In at least some embodiments, the shielding coil arrangement can include three separate coils or magnets to provide selectable shielding in all three dimensions. In at least some embodiments, the magnetic field generator 162 can include three orthogonal sets of coils to generate magnetic fields along three orthogonal axes. Other coil arrangement can also be used. The optional sensor(s) 164 can include, but are not limited to, one or more position sensors, orientation sensors, accelerometers, image recorders, or the like or any combination thereof.

The one or more magnetometers 160 can be any suitable magnetometer including, but not limited to, any suitable optically pumped magnetometer. Arrays of magnetometers are described in more detail herein. In at least some embodiments, at least one of the one or more magnetometers (or all of the magnetometers) of the system is arranged for operation in the SERF mode.

FIG. 1B is a schematic block diagram of one embodiment of a magnetometer 160 which includes a vapor cell 170 (also referred to as a “cell”) such as an alkali metal vapor cell; a heating device 176 to heat the cell 170; a light source 172; and a detector 174. In addition, coils of a magnetic field generator 162 can be positioned around the vapor cell 170. The vapor cell 170 can include, for example, an alkali metal vapor (for example, rubidium in natural abundance, isotopically enriched rubidium, potassium, or cesium, or any other suitable alkali metal such as lithium, sodium, or francium) and, optionally, one, or both, of a quenching gas (for example, nitrogen) and a buffer gas (for example, nitrogen, helium, neon, or argon). In some embodiments, the vapor cell may include the alkali metal atoms in a prevaporized form prior to heating to generate the vapor.

The light source 172 can include, for example, a laser to, respectively, optically pump the alkali metal atoms and probe the vapor cell. The light source 172 may also include optics (such as lenses, waveplates, collimators, polarizers, and objects with reflective surfaces) for beam shaping and polarization control and for directing the light from the light source to the cell and detector. Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source. In some embodiments, the light source 172 may include two light sources: a pump light source and a probe light source.

The detector 174 can include, for example, an optical detector to measure the optical properties of the transmitted probe light field amplitude, phase, or polarization, as quantified through optical absorption and dispersion curves, spectrum, or polarization or the like or any combination thereof. Examples of suitable detectors include, but are not limited to, a photodiode, charge coupled device (CCD) array, CMOS array, camera, photodiode array, single photon avalanche diode (SPAD) array, avalanche photodiode (APD) array, or any other suitable optical sensor array that can measure the change in transmitted light at the optical wavelengths of interest.

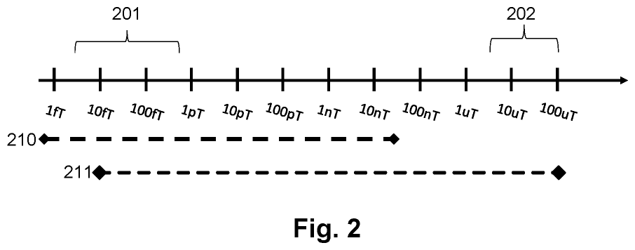

FIG. 2 shows the magnetic spectrum from 1 fT to 100 μT in magnetic field strength on a logarithmic scale. The magnitude of magnetic fields generated by the human brain are indicated by range 201 and the magnitude of the background ambient magnetic field, including the Earth's magnetic field, by range 202. The strength of the Earth's magnetic field covers a range as it depends on the position on the Earth as well as the materials of the surrounding environment where the magnetic field is measured. Range 210 indicates the approximate measurement range of a magnetometer (e.g., an OPM) operating in the SERF mode (e.g., a SERF magnetometer) and range 211 indicates the approximate measurement range of a magnetometer operating in a scalar mode (e.g., a scalar magnetometer.) Typically, a SERF magnetometer is more sensitive than a scalar magnetometer, but many conventional SERF magnetometers typically only operate up to about 0 to 200 nT while the scalar magnetometer starts in the 10 to 100 fT range but extends above 10 to 100 μT.

In at least some conventional magnetic field measurements systems, a magnetically shielded room is used to reduce the strength of the Earth field by 1,000 to 10,000 times. However, a passive, magnetically shielded room is often large, heavy, fixed, claustrophobic, and expensive. In addition, a single active coil system that can create a homogenous field region large enough to enable a cluster of OPM magnetometers that fit around the head of a user to simultaneously operate in a near-zero-field (NZF) environment would likely require coil supports so large as to preclude a wearable MEG system. Moreover, the relatively low coil efficiency (magnetic field per unit current) of such a large coil system may require large, high-current, low-noise coil driver electronics which can be expensive.

An alternative conventional method is to use electrical currents in specially shaped coils to actively counteract the ambient background magnetic field to create a small near-zero-field (NZF) environment surrounding the magnetically sensitive region of a single magnetometer or a small number of magnetometers. This arrangement provides active shielding. An analogy can be drawn to noise-canceling headphones that measure, then remove, unwanted noise by generating an inverse pressure waveform which cancels the noise.

A challenge with this architecture arises, however, when multiple near-zero-field (NZF) regions are constructed for a cluster of OPM magnetometers disposed around the head (or other region to be observed) to provide fuller head coverage of neural signals. The stray magnetic fields and magnetic field gradients emanating from the active shield surrounding each OPM magnetometer in the cluster (whether the active shield is associated with a single magnetometer, a magnetometer array or group, or a gradiometer) extend outward and substantially contaminate the magnetic environment of other OPM magnetometers elsewhere on the head. In the simplest case, where OPM magnetometers tile the head with a cluster of always-on actively-shielded magnetometers (or groups of magnetometers), the total stray fields of all of the active shields may hinder or prevent any single magnetometer from operating with sufficient sensitivity to observe, detect, or measure neural activity.

In contrast to these previous arrangements, in at least some embodiments, a system or method can interleave or multiplex operation of individual magnetometers (or groups of magnetometers) in a cluster, such that only a small number (for example, one, two, three, four, five, six, or more) of substantially non-interacting active shielding coil arrangements (such as the magnetic field generator 162 of FIGS. 1A and 1B) are actively operating at any one moment in time. As an example, an active shielding arrangement (such as the magnetic field generator 162 of FIGS. 1A and 1B) can have three shielding coils to provide controllable shielding in all three dimensions. By turning off some or most other active shielding arrangements momentarily, a reduced number of near-zero-field environments can be successfully created for a desired duration of time, enabling neural data from each location to be acquired in sequence. In at least some embodiments, this includes temporal multiplexing of the active shielding arrangements and their associated magnetometers. In at least some embodiments, adjustable duty cycle and spatial sequencing allows tuning a cluster of magnetometers for monitoring, observing, or measuring neural activity by interleaving or multiplexing operation of each OPM magnetometer demonstrating brain activity in its coverage domain.

Crosstalk between actively shielded sensors can result in reduced performance. The systems and methods described herein can reduce or prevent crosstalk by interleaving operation of the sensors and associated active shielding. Sharing other electrical components (for example, one or more of the following: ADCs, DACs, preamplifiers, laser drivers, thermistor drivers, of the power, data and signal processing pipeline for the active shields or magnetometers) can significantly reduce the complexity and weight of a wearable MEG device or other magnetic field measurements system.

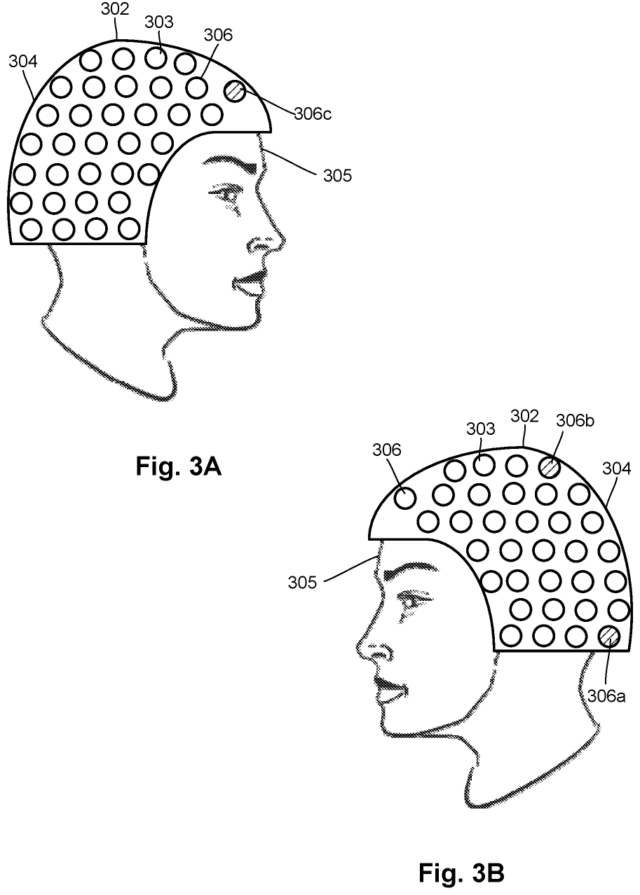

FIGS. 3A and 3B illustrate two sides of a wearable magnetic field measurement device 302 as worn by a user 305. The magnetic field measurement device 302 includes a body 304 and multiple sensors 303 (for example, OPM magnetometers, other magnetometers, or other magnetic field sensors) disposed on or in the body. The body 304 can take the form of, for example, a helmet, cap, hat, hood, scarf, wrap, or other headgear or any other suitable form, Further details discussing different form factors in small, portable, wearable devices and applications thereof are set forth in U.S. patent application Ser. Nos. 16/523,861 and 16/364,338, and U.S. Provisional Patent Applications Ser. Nos. 62/829,124; 62/839,405; 62/894,578; 62/859,880; and 62/891,128, all of which are incorporated herein by reference.

The sensors 303 can be arranged into multiple sensor units 306 with each sensor unit having one or more of the sensors. Each sensor unit 306 is associated with an active shield generated by an active shielding arrangement (for example, the magnetic field generator 162 of FIGS. 1A and 1B) which is part of the sensor unit. In at least some embodiments, such as the embodiment illustrated in FIGS. 3A and 3B, the sensor units 306 each include only a single sensor 303 with a different active shielding arrangement (for example, the magnetic field generator 162 of FIGS. 1A and 1B) associated with each of the sensors. In other embodiments, each sensor unit 306 can include one, two, three, four, or more sensors 303 with a single active shielding arrangement (for example, the magnetic field generator 162 of FIGS. 1A and 1B) associated with all of the sensors of that sensor unit. In at least some of these embodiments, the sensor units 306 may have different numbers of sensors 303. In at least some embodiments, the identity of location of individual sensor units 306 (and which sensors 303 are part of that sensor unit) may be selected based on functional regions of the brain or using any other suitable criteria.

In at least some embodiments, the active shielding arrangement can be defined by conductive coils (for example, copper traces on a printed circuit board or copper wires) which are capable, when energized, of cancelling or substantially reducing the ambient background magnetic field to create a localized region of near-zero-field (NZF) within which one or more sensors 303 (for example, one or more SERF-mode OPMs) can successfully operate for the detection of magnetic fields arising from neural activity (with signals often no more than tens of femtoTesla). For example, the ambient background magnetic field may be reduced to no more than 200, 100, 50, or 10 nT.

In at least some embodiments, the system or method may use multiple independent current sources for cancelation or reduction of the ambient background magnetic field or may use gradient (e.g., spatially varying magnetic fields) for cancelation or reduction of the ambient background magnetic field. In at least some embodiments, passive shielding (such as mu-metal passive shields) may be used in conjunction with the active shielding arrangement for reducing the ambient background magnetic field. Additional examples of passive shielding can be found in U.S. Provisional Patent Applications Ser. Nos. 62/719,928; 62/732,791; 62/776,895; 62/796,958; and 62/827,390 and U.S. patent applications Ser. Nos. 16/456,975 and 16/457,655, all of which are incorporated herein by reference.

Outside of the NZF region, however, the active shielding arrangement also typically generates large “stray” magnetic fields and magnetic field gradients which can hinder or prevent adjacent or nearby sensor units 306 from simultaneously operating to observe, measure, or detect neural activity. To reduce this additional source of magnetic fields, in some embodiments, a magnetic field measurement system is configured for operating each sensor unit 306 in a limited-duty-cycle mode in which the active shielding arrangement for one such sensor unit is turned on to enable its brief operation while substantially all of the other active shielding arrangement of the other sensor units are turned off.

In other embodiments, a magnetic field measurement system is configured for operating the active shielding arrangements of multiple sensor units (for example, sensor units 306a, 306b, and 306c in FIGS. 3A and 3B) simultaneously or in any other temporally overlapping manner so long as the active sensor units 306a, 306b, 306c are spaced apart from each other so that the corresponding active shielding arrangements do not generate substantial magnetic fields at the other active sensor units. In at least some embodiments, the active sensor units are selected so that, at each of the active sensor units, the combined magnitudes of the magnetic fields of all of the other active shielding arrangements is no more than 100, 50, 25, 10, 5, or 1 nT. In at least some embodiments, the active sensor units are selected so that at each of the active sensor units, the magnetic fields at of each of the other active shielding arrangements is reduced by at least a factor of at least 10,000; 8,000; or 5,000. In at least some embodiments, the active sensor units are selected so that at each of the active sensor units is at least 1, 2, 2.5, 3, 3.5, or 4 centimeters from any other active sensor unit. In at least some embodiments, the magnitude of a magnetic field generated by the active shielding arrangement of one active sensor unit at the site of another active sensor unit is less than the expected magnitude of the neural activity (or other biosignal) that is the object of observation.

Because the currents in the active shielding arrangements can be rapidly switched, as compared to the timescale of neural events, it is possible to interpolate the sensed neural magnetic fields for each sensor unit 306, albeit with reduced signal-to-noise ratio as determined by the duty cycle of each sensor unit 306 in the device, system, or cluster. Passive shielding may also reduce stray fields and gradients arising outside the NZF region of each sensor unit due to the active shielding arrangements of other sensor units. It will be recognized that a magnetic field measurement system can be configured to operate in any one (or all) of the described limited duty cycle modes, but may also be configured to operate in other modes, such as, for example, a mode in which more (or even all) of the active shielding arrangements are operating simultaneously.

In at least some embodiments, instead of the active shielding arrangement of each sensor unit 306 having its own power sources to provide the active shielding, a reduced number of current sources can be shared among the sensor units because of the temporal interleaving or multiplexing of the operation of the sensor units. Such embodiments may provide one or more of reduced design complexity, reduced cost, reduced power consumption, reduced weight and cabling, while maintaining the ability to observe and sense neural activity.

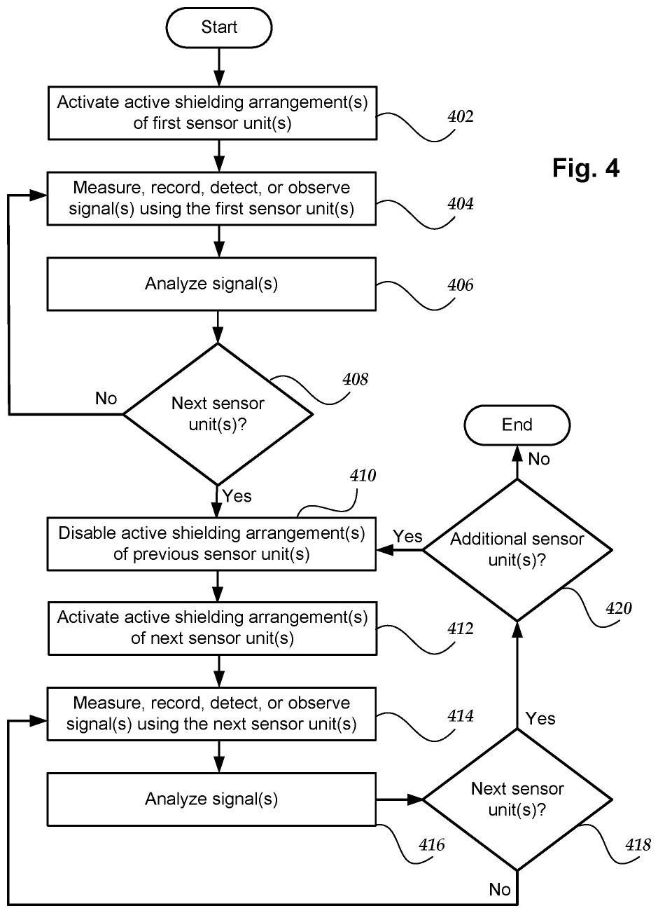

FIG. 4 is a flowchart illustrating one embodiment of a method or system which utilizes the interleaving or multiplexing of sensor unit operation in a limited duty cycle mode, as described above. In step 402, the active shielding arrangement(s) of one or more first sensor units is enabled and activated to produce a magnetic field and provide an NZF region around the first sensor unit(s). If the active shielding arrangements of multiple sensor units are enabled and activated in step 402 (or step 412), these sensor units are preferably spaced sufficiently apart so that the magnetic fields generated by the active shielding arrangements of other sensor units are smaller than the expected magnetic fields from the neural activity or other biosignal that is to be detected.

In step 404, the first sensor unit(s) measure, record, detect, or otherwise observe signal(s) such as, for example, the magnetic fields arising from neural activity. In step 406, the signals are analyzed. For example, the signals may be analyzed to determine if any neural activity (or a threshold amount of neural activity) is measured, recorded, detected, or otherwise observed by the first sensor unit(s).

In step 408, it is determined whether to switch to the next sensor unit(s). If not, then steps 404, 406, and 408 are repeated. If so, then the method or system proceeds to step 410. This determination can be made based on any suitable criteria. For example, the system or method may automatically switch to the next sensor unit(s) after the measurements and analysis. In other embodiments, the determination may be made based on the analysis in step 406. For example, if neural activity is measured, recorded, detected, or otherwise observed by the first sensor unit(s) then the system or method may repeat steps 404, 406, and 408 to measure, record, detect, or otherwise observe ongoing neural activity. Alternatively, the detected neural activity using the first sensor unit(s) may lead the method of system to move to other sensor unit(s) that are likely to also measure, record, detect, or otherwise observe neural activity. If no neural activity (or neural activity below a threshold amount) is measured, recorded, detected, or otherwise observed by the first sensor unit(s), then the determination in step 408 may be to continue to the next sensor unit(s).

In at least some embodiments, the method or system can include dynamic control of the duty cycle and spatially specific activation of sensor units to acquire high signal-to-noise data with specific correlation to the underlying brain activity for a given neural task. In at least some embodiments, the system or method may identify the information content arising from a single area of a user's brain and the system or method may include deciding whether to switch to a new area or continue to record or sense in the current region. Adaptive algorithms can dynamically adjust the active sensor unit or subset of sensor units to match changing neural field patterns, both spatially and temporally. In at least some embodiments, a single substantially-full-head coverage system can be dynamically reconfigured for selective use over different regions of the brain. In at least some embodiments, a temporally multiplexed system or method may enable dynamic reconfiguration of the control electronics to emphasize power savings when appropriate, or high signal to noise in a localized region, or correlation between specific localized regions, or the like or any combination thereof. In at least some embodiments, the method of system can include flexible adjustment of MEG coverage for different users without necessarily changing the physical configuration of the sensor array.

In step 410, the active shielding arrangement(s) of the previous sensor unit(s) (e.g., the first sensor unit(s) after execution of step 408) is disabled. Steps 412, 414, 416, and 418 are the same as steps 402, 404, 406, and 408, respectively, except that the next sensor unit(s) is used. In step 420, a determination is made whether there are any additional sensor unit(s) to measure signal(s). If yes, then the method or system returns to step 410. If no, then the procedure terminates.

None of steps 408, 418, and 420 preclude returning to a sensor unit that has already been used in preceding steps. As an example, a procedure may first use a first sensor unit in steps 402 to 410, then a second sensor unit in steps 412 to 420 and the repeated instance of step 410, then a third sensor unit in the following steps 412 to 420 and the next instance of step 410, and then return to the first sensor unit in another loop of steps 412 to 420 and step 410.

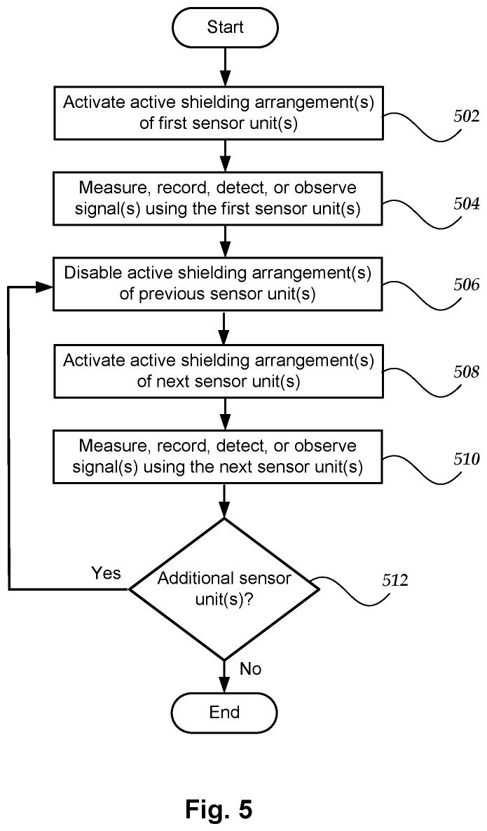

FIG. 5 illustrates another embodiment of a method or system which utilizes the interleaving or multiplexing of sensor unit operation in a limited duty cycle mode, as described above. In this embodiment, however, the analysis of the signal(s) is not used to determine whether to proceed with the next sensor unit(s) or to select the next sensor unit(s). For example, the temporal arrangement of sensor unit(s) may be fixed or programmed by the manufacturer, user, or other individual. In at least some embodiments, the method or system may simply cycle through all of the sensor unit(s) in a fixed or programmable order. As in the previous embodiment, however, the system and method are not precluded from returning to a sensor unit(s) that has already been used in preceding steps.

In step 502, the active shielding arrangement(s) of one or more first sensor units is enabled and activated to produce a magnetic field and provide an NZF region around the first sensor unit(s). IF the active shielding arrangements of multiple sensor units are enabled and activated in step 502 (or step 508), these sensor units are preferably spaced sufficiently apart so that the magnetic fields generated by the active shielding arrangements of other sensor units are smaller than the magnetic fields from the neural activity that is to be detected.

In step 504, the first sensor unit(s) measure, record, detect, or otherwise observe signal(s) such as, for example, the magnetic fields arising from neural activity. In step 506, the active shielding arrangement(s) of the previous sensor unit(s) (e.g., the first sensor unit(s) after execution of step 504) is disabled. Steps 508 and 510 are the same as steps 502 and 504, respectively, except that the next sensor unit(s) are used. In step 512, a determination is made whether there are any additional sensor unit(s) to measure signal(s). If yes, then the method or system returns to step 506. If no, then the procedure terminates.

Examples of magnetic field measurement systems in which the embodiments presented above can be incorporated, and which present features that can be incorporated in the embodiments presented herein, are described in U.S. Patent Application Publications Nos. 2020/0072916; 2020/0056263; 2020/0025844; 2020/0057116; 2019/0391213; 2020/0088811; 2020/0057115; 2020/0109481; 2020/0123416; and 2020/0191883; U.S. patent applications Ser. Nos. 16/741,593; 16/752,393; 16/820,131; 16/850,380; 16/850,444; 16/884,672; 16/904,281; 16/922,898; and 16/928,810, and U.S. Provisional Patent Applications Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; 62/983,406; 63/031,469; and 63/037,407, all of which are incorporated herein by reference.

The above specification provides a description of the invention and its manufacture and use. Since many embodiments of the invention can be made without departing from the spirit and scope of the invention, the invention also resides in the claims hereinafter appended.

What is claimed as new and desired to be protected by Letters Patent of the United States is:

1. A magnetic field measurement system for measuring biosignals, comprising:
a body;
a plurality of sensors units, each of the sensor units comprising at least one magnetic field sensor disposed on or in the body;
a plurality of magnetic field generators, each of the magnetic field generators associated with a different one of the sensor units, wherein each of the magnetic field generators is configured to provide active shielding to the associated sensor unit when the magnetic field generator is activated; and
a processor coupled to the magnetic field sensors and the magnetic field generators, wherein the processor is configured to perform actions comprising:
1) selecting at least one of the sensor units, wherein, when multiple sensor units are selected, the selected sensor units are spatially separated from each other;
2) for each of the at least one selected sensor unit, activating the magnetic field generator associated with that selected sensor unit to provide active shielding;
3) receiving signals from the at least one selected sensor unit; and
4) repeating 1) through 3) at least once.

2. The magnetic field measurement system of claim 1, wherein the actions further comprise disabling the magnetic field generator associated with the at least one selected sensor unit after receiving the signals from the at least one selected sensor unit.

3. The magnetic field measurement system of claim 1, wherein repeating 1) through 3) comprises 1) through 3) until a programmed termination is reached by the system.

4. The magnetic field measurement system of claim 1, wherein repeating 1) through 3) comprises 1) through 3) until termination by the user.

5. The magnetic field measurement system of claim 1, wherein, when multiple sensor units are selected, the selected sensor units are spatially separated from each other so that, at each of the selected sensor units, a combined magnitude of the magnetic fields of all of the active shielding arrangements of the other selected sensor units is no more than 50 nT.

6. The magnetic field measurement system of claim 1, wherein, when multiple sensor units are selected, the selected sensor units are spatially separated from each other by at least 2 centimeters.

7. The magnetic field measurement system of claim 1, wherein each of the sensor units consists of a single one of the magnetic field sensors.

8. The magnetic field measurement system of claim 1, wherein each of the magnetic field sensors is an optically pumped magnetometer.

9. The magnetic field measurement system of claim 1, further comprising passive shielding disposed in the body to provide shielding for at least one of the sensor units.

10. The magnetic field measurement system of claim 1, wherein selecting at least one of the sensor units comprising selecting only one of the sensor units.

11. The magnetic field measurement system of claim 1, wherein, upon repeating 1) through 3), selecting at least one of the sensor units comprises selecting at least one of the sensor units based on the received signals.

12. The magnetic field measurement system of claim 1, wherein, upon repeating 1) through 3), selecting at least one of the sensor units comprises selecting at least one of the sensor units based on a predetermined order.

13. The magnetic field measurement system of claim 1, wherein the actions further comprise analyzing the received signals.

14. The magnetic field measurement system of claim 1, wherein the body is a wearable device.

15. A non-transitory computer-readable medium having stored thereon instructions for execution by a processor to perform actions including:
1) selecting at least one of a plurality of sensor units of a wearable device of a magnetic field measurement system, wherein, when multiple sensor units are selected, the selected sensor units are spatially separated from each other;
2) for each of the at least one selected sensor unit, activating a magnetic field generator associated with that selected sensor unit to provide active shielding;
3) receiving signals from the at least one selected sensor unit; and
4) repeating 1) through 3) at least once.

16. The non-transitory computer-readable medium of claim 15, wherein the actions further comprise disabling the magnetic field generator associated with the at least one selected sensor unit after receiving the signals from the at least one selected sensor unit.

17. The non-transitory computer-readable medium of claim 15, wherein the actions further comprise analyzing the received signals.

18. A method for monitoring biologically generated magnetic fields, the method comprising:
1) selecting at least one of a plurality of sensor units of a wearable device of a magnetic field measurement system, wherein, when multiple sensor units are selected, the selected sensor units are spatially separated from each other;
2) for each of the at least one selected sensor unit, activating a magnetic field generator associated with that selected sensor unit to provide active shielding;
3) receiving signals from the at least one selected sensor unit; and
4) repeating 1) through 3) at least once.

19. The method of claim 18, further comprising disabling the magnetic field generator associated with the at least one selected sensor unit after receiving the signals from the at least one selected sensor unit.

20. The method of claim 18, further comprising analyzing the received signals.
